(19) United States
(12) Patent Application Publication
(10) Pub. No.: US 2026/0207923 A1
(43) Pub. Date: Jul. 23, 2026

(54) SYSTEMS AND METHODS FOR THERMAL MITIGATION IN MEDICAL DEVICE CONTROLLERS

(71) Applicant: TC1 LLC, St. Paul, MN (US)

(72) Inventors: John Freddy Hansen, Livermore, CA (US); Elizabeth Afanasewicz, Allston, MA (US)

(21) Appl. No.: 19/140,774

(22) PCT Filed: Dec. 18, 2023
(86) PCT No.: PCT/US2023/084598

§ 371 Date: Jun. 18, 2025

Related U.S. Application Data
(60) Provisional application No. 63/434,689, filed on Dec. 22, 2022.

(51) Int. Cl. A61M 60/585

(52) U.S. Cl. CPC A61M 60/585; A61M 2205/3606

(57) ABSTRACT

An external controller device for operating a medical device implanted within a patient is provided. The external controller device includes a housing including at least a top wall and a bottom wall, wherein the bottom wall is configured to be closer to the patient than the top wall during operation of the external controller device, at least one heat generating component positioned within the housing and configured to control the operation of the external controller device, and a dividing wall positioned between the top wall and the bottom wall, wherein a first compartment is defined between the dividing wall and the top wall, wherein a second compartment is defined between the dividing wall and the bottom wall, and wherein the at least one heat generating component is positioned within the first compartment.

### CROSS-REFERENCE TO RELATED APPLICATIONS

This application claims priority to U.S. Provisional Application No. 63/434,689, filed on Dec. 22, 2022, the entire contents of which are hereby incorporated herein by reference.

### BACKGROUND OF THE DISCLOSURE

### a. Field of the Disclosure

The present disclosure relates generally to implantable medical devices, and more specifically, relates to thermal mitigation for external controllers of implantable medical devices.

### b. Background

There are various types of implantable medical devices, including pacemakers and heart pumps. These devices may be controlled, at least in part, using a controller device that is external to the patient. The controller device may be worn by the patient (e.g., on a harness or belt, or in a pocket of a garment). Accordingly, the controller device may be in close proximity to the patient and may contact the patient.

During operation, electronic components within the controller device may generate thermal energy (i.e., heat). Given the proximity of the controller device to the patient, it would be desirable to reduce the thermal energy transferred to the patient from the controller device.

### SUMMARY OF THE DISCLOSURE

In one aspect, an external controller device for operating a medical device implanted within a patient is provided. The external controller device includes a housing including at least a top wall and a bottom wall, wherein the bottom wall is configured to be closer to the patient than the top wall during operation of the external controller device, at least one heat generating component positioned within the housing and configured to control the operation of the external controller device, and a dividing wall positioned between the top wall and the bottom wall, wherein a first compartment is defined between the dividing wall and the top wall, wherein a second compartment is defined between the dividing wall and the bottom wall, and wherein the at least one heat generating component is positioned within the first compartment.

In another aspect, an external controller device for operating a medical device implanted within a patient is provided. The external controller device includes a housing including at least a top wall and a bottom wall, wherein the bottom wall is configured to be closer to the patient than the top wall during operation of the external controller device at least one heat generating component positioned within the housing and configured to control the operation of the external controller device, and a heat spreader configured to spread thermal energy generated by the at least one heat generating component over the top wall.

In yet another aspect, a method of assembling an external controller device for operating a medical device implanted within a patient is provided. The method includes forming a housing including at least a top wall and a bottom wall, wherein the bottom wall is configured to be closer to the patient than the top wall during operation of the external controller device positioning a dividing wall between the top wall and the bottom wall, wherein a first compartment is defined between the dividing wall and the top wall, and wherein a second compartment is defined between the dividing wall and the bottom wall, and positioning at least one heat generating component within the first compartment, the at least one heat generating component configured to control the operation of the external controller device.

### BRIEF DESCRIPTION OF THE DRAWINGS

FIG. 1 is a simplified electrical circuit diagram of one embodiment of a wireless power transfer system.

FIG. 2 is an illustration of the wireless power transfer system of FIG. 1 being used to supply power to a ventricular assist device (VAD).

FIG. 3 is a front perspective view of components of one embodiment of a wireless power transfer system.

FIG. 4 is a side schematic view of an external controller device that may be used with the system shown in FIG. 3.

### DETAILED DESCRIPTION OF THE DISCLOSURE

The present disclosure is directed to an external controller device for operating a medical device implanted within a patient. The external controller device includes a housing including at least a top wall and a bottom wall, wherein the bottom wall is configured to be closer to the patient than the top wall during operation of the external controller device, at least one heat generating component positioned within the housing and configured to control the operation of the external controller device, and a dividing wall positioned between the top wall and the bottom wall, wherein a first compartment is defined between the dividing wall and the top wall, wherein a second compartment is defined between the dividing wall and the bottom wall, and wherein the at least one heat generating component is positioned within the first compartment.

As used herein, a “controller” or “controller device” refers to any device that supports operation of an implantable medical device. For example, a “controller” or “controller device”, as used herein, includes devices that actively control operation of an implantable medical device, as well as devices that power an implantable medical device (e.g., a power supply or battery pack).

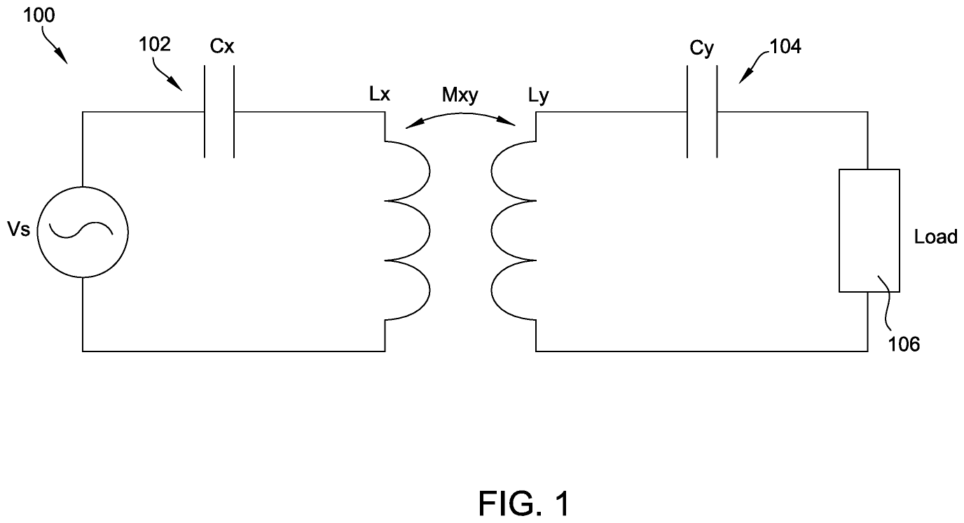

Referring now to the drawings, FIG. 1 is a simplified circuit of an example wireless power transfer system 100. The system 100 includes an external transmit resonator 102 and an implantable receive resonator 104. In the system shown in FIG. 1, a power source Vs is electrically connected with the transmit resonator 102, providing power to the transmit resonator 102. The receive resonator 104 is connected to a load 106 (e.g., an implantable medical device). The receive resonator 104 and the load 106 may be electrically connected with a switching or rectifying device (not shown).

In the example embodiment, the transmit resonator 102 includes a coil Lx connected to the power source Vs by a capacitor Cx. Further, the receive resonator 104 includes a coil Ly connected to the load 106 by a capacitor Cy. Inductors Lx and Ly are coupled by a coupling coefficient k. M_{xy} is the mutual inductance between the two coils. The mutual inductance, M_{xy}, is related to the coupling coefficient k as shown in the below Equation (1).

$${M}_{x y} = k \sqrt{{L}_{x} \cdot {L}_{y}} \qquad ( 1 )$$

In operation, the transmit resonator 102 transmits wireless power received from the power source Vs. The receive resonator 104 receives the power wirelessly transmitted by the transmit resonator 102, and transmits the received power to the load 106.

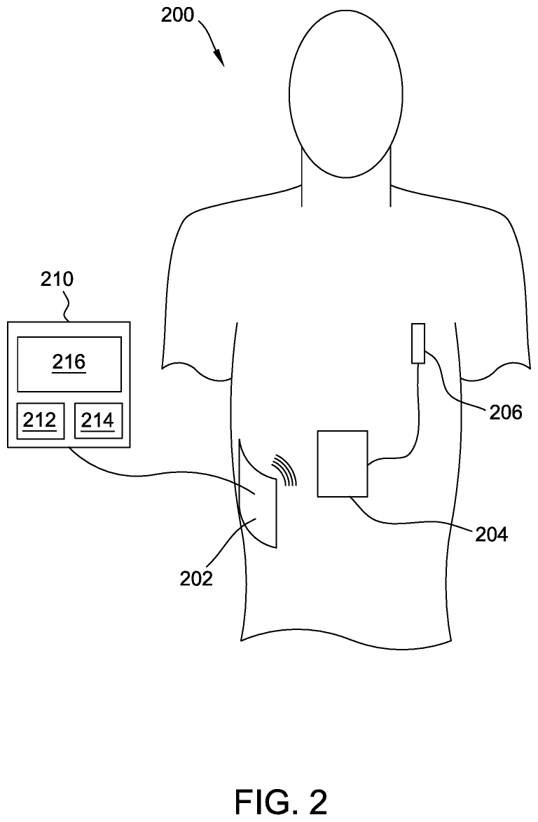

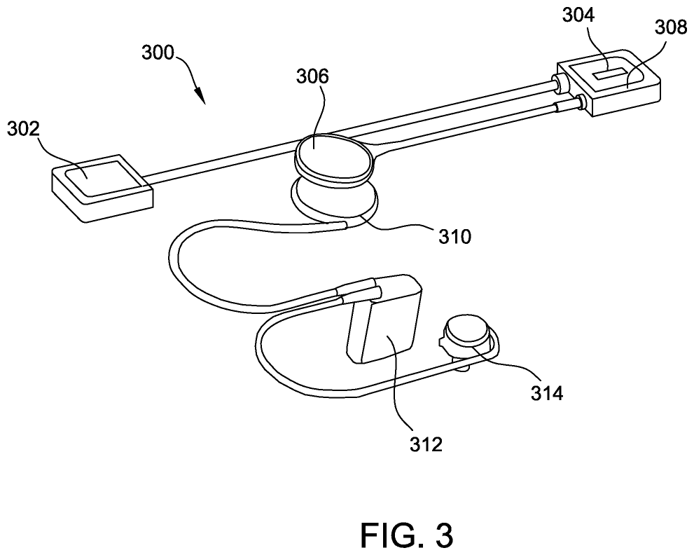

FIG. 2 illustrates one embodiment of a patient 200 using an external coil 202 (such as the transmit resonator 102 shown in FIG. 1) to wirelessly transmit power to an implanted coil 204 (such as the receive resonator shown in FIG. 1). The implanted coil 204 uses the received power to power an implanted device 206. For example, the implanted device 206 may include a pacemaker or heart pump (e.g., a left ventricular assist device (LVAD)). In some embodiments, the implanted coil 204 and/or the implanted device 206 may include or be coupled to a controller or a battery (not shown in FIG. 2). For example, in some embodiments, a controller and/or a battery may be coupled between the implanted coil 204 and the implanted device 206 (see, e.g., FIG. 3).

In one embodiment, the external coil 202 is communicatively coupled to a computing device 210, for example, via wired or wireless connection, such that the external coil 202 may receive signals from and transmit signals to the computing device 210. In some embodiments, the computing device 210 is a power source for the external coil 202. In other embodiments, the external coil 202 is coupled to an alternative power supply (not shown). The computing device 210 includes a processor 212 in communication with a memory 214. In some embodiments, executable instructions are stored in the memory 214.

The computing device 210 further includes a user interface (UI) 216. The UI 216 presents information to a user (e.g., the patient 200). For example, the UI 216 may include a display adapter (not shown) that may be coupled to a display device, such as a cathode ray tube (CRT), a liquid crystal display (LCD), an organic LED (OLED) display, and/or an “electronic ink” display. UI 216 may also include one or more user interfaces that a user may interact with (e.g., buttons, membrane switches, touch screen, etc.). In some embodiments, the UI 216 includes one or more display devices. Further, in some embodiments, presentation interface may not generate visual content, but may be limited to generating audible and/or computer-generated spoken-word content. In the example embodiment, the UI 216 displays one or more representations designed to aid the patient 200 in placing the external coil 202 such that the coupling between the external coil 202 and the implanted coil 204 is optimal. In some embodiments, the computing device 210 may be a wearable device. For example, in one embodiment, the computing device 210 is a wrist watch, and the UI 216 is displayed on the wrist watch.

FIG. 3 is a perspective view of components of one embodiment of a wireless power transfer system 300. System includes a battery pack 302, a hub device 304 (e.g., the computing device 210 shown in FIG. 2), and a transmitter 306 (e.g., the external coil 202 shown in FIG. 2). The hub device 304 is coupled between the battery pack 302 and the transmitter 306. Further, the hub device 304 includes a user interface (UI) 308 that enables a user to operate the wireless power transfer system 300. For example, the user may control the amount of power supplied from the battery pack 302 to the transmitter 306 using the UI 308. The wireless power transfer system 300 further includes an implanted receiver 310 (e.g., the implanted coil 204 shown in FIG. 2), a controller 312, and an implanted device 314 (e.g., the implanted device 206 shown in FIG. 2). The implanted device 314 may be, for example, a heart pump. The controller 312 is coupled between the implanted device 314 and the receiver 310. The controller 312 controls operation of the implanted device 314 (e.g., by controlling power delivery from the receiver 310 to the implanted device 314).

The battery pack 302, the hub device 304, and the transmitter 306 are located outside a patient's body (e.g., the patient 200, shown in FIG. 2) during operation of the wireless power transfer system 300. In contrast, the receiver 310, the controller 312, and the implanted device 314 are implanted within the patient's body during operation of the wireless power transfer system 300. During operation, the transmitter 306 wirelessly transmits radio-frequency (RF) power to the receiver 310.

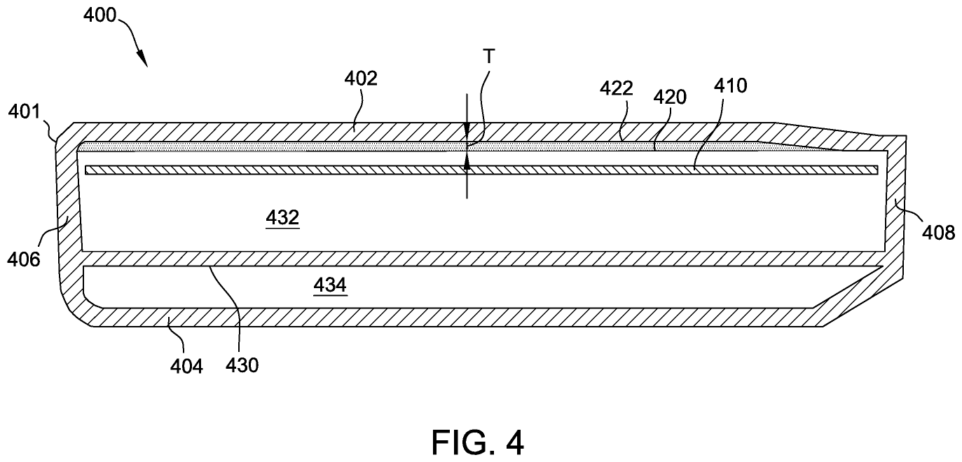

FIG. 4 is a side schematic view of an external controller device 400 for controlling operation of an implantable medical device, such as the implanted device 314 (shown in FIG. 3). Controller device 400 may be, for example, the hub device 304 (shown in FIG. 3). Alternatively, the controller device 400 may be any suitable device.

The controller device 400 has a housing 401 formed from a top wall 402, a bottom wall 404, a first sidewall 406 extending between the top and bottom walls 402 and 404, and a second sidewall 408 extending between the top and bottom walls 402 and 404. The housing 401 also includes first and second end walls (not shown in FIG. 4) that are oriented generally perpendicular to the top wall 402, the bottom wall 404, the first sidewall 406, and the second sidewall 408. In this embodiment the housing 401 is generally shaped as a rectangular prism. Alternatively, the housing 401 may have any suitable shape.

The various walls of the housing 401 may all be made from the same material, or one or more of the walls may be made of different materials. Further, in some embodiments, at least one of the walls (e.g., the top wall, the bottom wall 404, the first sidewall 406, and/or the second sidewall 408) may be selectively removable from the rest of the housing 401 to facilitate assembling and/or servicing the controller device 400 (e.g., by facilitating access to components inside the housing 401).

In some embodiments, at least one electronics component inside the housing 401 (e.g., the heat generating component 410 discussed below) may be attached to one of the walls, such that the electronics component slides out of the housing 401 with that wall when that wall is removed from the housing 401.

To facilitate controlling an implantable medical device, the controller device 400 may be worn by the patient (e.g., on a harness or belt, or in a pocket of a garment). In one embodiment, while being worn, the controller device 400 is positioned such that the bottom wall 404 faces the patient and the top wall 402 faces outward, away from the patient. That is, the bottom wall 404 of the controller device 400 is closer to the patient's skin than the top wall 402. In some instances, the bottom wall 404 may contact the patient's skin.

As described herein, the controller device 400 includes one or more thermal energy mitigation features to facilitate reducing thermal energy directed to the patient from the controller device 400. A number of different thermal energy mitigation features are described herein, and those of skill in the art will appreciate that different combinations of the thermal energy mitigation features may be implemented in different embodiments. Further, in some embodiments, only one of the thermal energy mitigation features may be used.

One example of a thermal energy mitigation feature includes positioning a heat generating component 410 within the controller device 400 to facilitate reducing thermal energy directed to the patient. As shown in FIG. 4, the heat generating component 410 is positioned proximate to the top wall 402 and is positioned remotely from the bottom wall 404. Accordingly, thermal energy generated by the heat generating component 410 generally leaves the controller device through the top wall 402 that faces away from the patient, not the bottom 404 that faces towards the patient. The heat generating component 410 may be, for example, an electronic component that facilitates controlling operation of the controller device 400. For example, the heat generating component may be a printed circuit board (PCB) including circuitry for operating the controller device 400.

Another example of a thermal energy mitigation feature is a heat spreader 420 in the controller device 400. In FIG. 4, the heat spreader 420 is positioned in contact with an inner surface 422 of the top wall 402. Here, the heat spreader 420 covers the entire inner surface 422 to facilitate spreading thermal energy over the entirety of top wall 402, reducing the overall average temperature of the controller device 400. Alternatively, the heat spreader 420 may only cover a portion of the inner surface 422. Further, in some embodiments, the heat spreader 420 may additionally or alternatively cover at least a portion of the first sidewall 406, the second sidewall 408, the first end wall, and/or the second end wall to facilitate spreading thermal energy over those walls.

The heat spreader 420 is fabricated from a material with a relatively high thermal conductivity (e.g., copper or aluminum). A thickness, T, of the heat spreader 420 may be selected based on the material of the heat spreader 420, the geometry of the controller device 400, and/or the total heat loads generated inside the controller device 400. For example, a copper heat spreader may have a thickness in a range from approximately 0.25 millimeters (mm) to 1.0 mm. Further, an aluminum heat spreader may have a thickness in a range from approximately 0.5 mm to 2.0 mm. Alternatively, the heat spreader 420 may be fabricated from any suitable material (e.g., graphite, gold, stainless steel, beryllium, etc.) and may have any suitable dimensions.

In some embodiments, instead of the heat spreader 420 being in contact with the top wall 402, the top wall 402 itself may be fabricated from a material that enables the top wall 402 to function as a heat spreader. In this situation, the top wall 402 may include one or more heat spreading features (e.g., grooves, holes, pins, ridges, etc.) that increase the surface area of the heat spreader without necessarily increasing the overall dimensions of the controller device 400. Alternatively or additionally, at least one of the first sidewall 406, the second sidewall 408, the first end wall, and/or the second end wall may be fabricated from a material to function as a heat spreader.

In the embodiment shown, the heat spreader 420 is a single plate of material. Alternatively, the heat spreader 420 may have any suitable configuration. For example, in some embodiments, the heat spreader 420 includes a thermal insulator (e.g., aerogel, Poron, epoxy, plastic, Kapton, or Mylar) positioned between two layers of a thermally conductive material (e.g., copper, aluminum, graphite, gold, stainless steel, beryllium, etc.). Each layer of thermally conductive material conducts heat well in the plane of that layer, and the thermal insulation slows the transfer of thermal energy between the two layers of thermally conductive material. In other embodiments, the heat spreader includes additional layers of thermally conductive material and thermal insulators.

Further, in some embodiments, a thermal interface material is positioned between the heat spreader 420 and at least one heat generating component, and contacts both the heat spreader 420 and the at least one heat generating component. Accordingly, thermal energy is conducted from the at least one heat generating component to the heat spreader 420 though the thermal interface material.

Yet another example of a thermal energy mitigation feature is a dividing wall 430 in the controller device 400. The dividing wall 430 divides an interior volume of the controller device into a first compartment 432 and a second compartment 434. The dividing wall 430 may be made of any suitable material. For example, the dividing wall 430 may be made of a metal and/or a plastic (e.g., a polycarbonate). The first compartment 432 is defined between the top wall 402 and the dividing wall 430, and the second compartment 434 is defined between the bottom wall 404 and the dividing wall 430. Thus, when the controller device 400 is worn by a patient, the second compartment 434 is positioned between the first compartment 432 and the patient. In the embodiment shown, the first compartment 432 has a larger volume than the second compartment 434. Alternatively, the first compartment 432 may have a smaller volume than the second compartment 434.

In some embodiments, the bottom wall 404 and/or the dividing wall 430 are selectively removable from the rest of the housing 401 (e.g., to provide access to the first compartment 432 and/or the second compartment 434). In one embodiment, the bottom wall 404, the dividing wall 430, and portions of the first and second end walls and the first and second sidewalls 406 and 408 extending between the bottom wall 404 and the dividing wall 430 are removable as a unit or module from the rest of the housing 401. Thus, the second compartment 434 functions as a module that can be selectively removed from or added to the controller device 400. In at least some of the embodiments, at least one electrical component may be attached to the removable wall(s) to slide out from the housing 401 with the removable wall(s).

To improve thermal energy mitigation, all heat generating components for the controller device 400 are located in the first compartment 432, away from the patient. For example, as shown in FIG. 4, the heat generating component 410 is positioned within the first compartment 432. The second compartment 434, in contrast, does not contain any heat generating components. This arrangement prevents hot air generated inside the first compartment 432 from transferring thermal energy into the second compartment 434 (e.g., by convection) and heating the patient.

The second compartment 434 may include a gaseous, solid, and/or liquid material therein. For example, in one embodiment, the second compartment 434 includes a material with a relatively low thermal conductivity (e.g., an aerogel). In other embodiments, the second compartment 434 includes a gas (e.g., air, helium, argon, and/or nitrogen). Further, in some embodiments, a gas (e.g., helium and/or argon) is included in the first compartment 432.

The first compartment 432 is typically hermetically sealed to prevent water and/or dust from entering the compartment 432 and interfering with the operation of the components therein. In contrast, in some embodiments, the second compartment 432 is not hermetically sealed. For example, one or more apertures (e.g., holes or slots) may be defined in the first sidewall 406, the second sidewall 408, the first end wall, and/or the second end wall to allow airflow into and out of the second compartment 432.

Implementing one or more of the thermal energy mitigation features described herein facilitates reducing the thermal energy experienced by a patient wearing the controller device 400. For example, in some embodiments, the temperature experienced by the patient may be reduced by 1° to 3° Celsius, which is a substantial improvement.

Further, increasing the surface area of a device also facilitates reducing the temperature. Removing heat generating components also reduces the temperature. For example, relocating components from the controller device 400 to another device in the wireless power transfer system 300 (e.g., battery pack 302) and/or increasing the surface area of the controller device 400 may improve thermal performance by at least 75%.

Although the embodiments described herein are discussed in the context of a wireless power transfer system for a heart pump, those of skill in the art will appreciate that the thermal mitigation features described herein may be implemented in external controllers for a variety of implantable medical devices.

The embodiments described herein are directed an external controller device for operating a medical device implanted within a patient. The external controller device includes a housing including at least a top wall and a bottom wall, wherein the bottom wall is configured to be closer to the patient than the top wall during operation of the external controller device, at least one heat generating component positioned within the housing and configured to control the operation of the external controller device, and a dividing wall positioned between the top wall and the bottom wall, wherein a first compartment is defined between the dividing wall and the top wall, wherein a second compartment is defined between the dividing wall and the bottom wall, and wherein the at least one heat generating component is positioned within the first compartment.

Although the embodiments and examples disclosed herein have been described with reference to particular embodiments, it is to be understood that these embodiments and examples are merely illustrative of the principles and applications of the present disclosure. It is therefore to be understood that numerous modifications can be made to the illustrative embodiments and examples and that other arrangements can be devised without departing from the spirit and scope of the present disclosure as defined by the claims. Thus, it is intended that the present application cover the modifications and variations of these embodiments and their equivalents.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An external controller device for operating a medical device implanted within a patient, the external controller device comprising:
a housing comprising at least a top wall and a bottom wall, wherein the bottom wall is configured to be closer to the patient than the top wall during operation of the external controller device;
at least one heat generating component positioned within the housing and configured to control the operation of the external controller device; and
a dividing wall positioned between the top wall and the bottom wall, wherein a first compartment is defined between the dividing wall and the top wall, wherein a second compartment is defined between the dividing wall and the bottom wall, and wherein the at least one heat generating component is positioned within the first compartment.

2. The external controller device of claim 1, further comprising a heat spreader configured to spread thermal energy generated by the at least one heat generating component over at least one wall of the housing.

3. The external controller device of claim 2, wherein the heat spreader is fabricated from copper, aluminum, graphite, gold, stainless steel, and/or beryllium.

4. The external controller device of claim 2, wherein the heat spreader is in contact with an inner surface of the at least one wall.

5. The external controller device of claim 2, wherein the heat spreader is incorporated within at least one wall.

6. The external controller device of claim 1, wherein the at least one heat generating component is positioned proximate to the top wall.

7. The external controller device of claim 1, wherein the second compartment includes a gaseous material.

8. The external controller device of claim 1, wherein the housing defines at least one aperture that allows airflow into and out of the second compartment.

9. The external controller device of claim 1, wherein the second compartment includes an aerogel.

10. The external controller device of claim 1, wherein the external controller device is configured to control operation of an implantable blood pump.

11. An external controller device for operating a medical device implanted within a patient, the external controller device comprising:
a housing comprising at least a top wall and a bottom wall, wherein the bottom wall is configured to be closer to the patient than the top wall during operation of the external controller device;
at least one heat generating component positioned within the housing and configured to control the operation of the external controller device; and
a heat spreader configured to spread thermal energy generated by the at least one heat generating component over the top wall.

12. The external controller device of claim 11, wherein the at least one heat generating component is positioned proximate to the top wall.

13. The external controller device of claim 11, further comprising:
a dividing wall positioned between the top wall and the bottom wall, wherein a first compartment is defined between the dividing wall and the top wall, wherein a second compartment is defined between the dividing wall and the bottom wall, and wherein the at least one heat generating component is positioned within the first compartment.

14. The external controller device of claim 13, wherein the second compartment includes a gaseous material.

15. The external controller device of claim 13, wherein the housing defines at least one aperture that allows airflow into and out of the second compartment.

16. The external controller device of claim 13, wherein the second compartment includes an aerogel.

17. The external controller device of claim 11, wherein the heat spreader is fabricated from copper, aluminum, graphite, gold, stainless steel, and/or beryllium.

18. The external controller device of claim 11, wherein the heat spreader is in contact with an inner surface of the top wall.

19. The external controller device of claim 11, wherein the heat spreader is incorporated within the top wall.

20. A method of assembling an external controller device for operating a medical device implanted within a patient, the method comprising:
forming a housing including at least a top wall and a bottom wall, wherein the bottom wall is configured to be closer to the patient than the top wall during operation of the external controller device;
positioning a dividing wall between the top wall and the bottom wall, wherein a first compartment is defined between the dividing wall and the top wall, and wherein a second compartment is defined between the dividing wall and the bottom wall; and
positioning at least one heat generating component within the first compartment, the at least one heat generating component configured to control the operation of the external 10 controller device.
